(19) United States
(12) Patent Application Publication
(10) Pub. No.: US 2026/0337242 A1
(43) Pub. Date: Oct. 29, 2026

(54) ENRICHMENT OF A CASEIN IN A COMPOSITION

(71) Applicant: NEWMILKBUZZ B.V., Etten-Leur (NL)

(72) Inventors: Niels DAELMAN, Kortrijk (BE); Franck Philibert Noël FUDALEJ, Rueil-Malmaison (FR); Edward Hendrik KREKELBERGH, Gent (BE); Kathleen Camilla Telesphore Alida Maria PIENS, Gent (BE); Gerardus Johannes Franciscus SMOLDERS, Delft (NL); Erik DE VROOM, Leiden (NL)

(73) Assignee: NEWMILKBUZZ B.V., Etten-Leur (NL)

(21) Appl. No.: 19/159,644

(22) PCT Filed: Mar. 8, 2024
(86) PCT No.: PCT/EP2024/056119

§ 371 Date: Aug. 26, 2025

(30) Foreign Application Priority Data
Mar. 10, 2023 (EP) 23161294.6

(51) Int. Cl. C07K 14/47; A23C 19/055; A23C 20/00; A23J 1/00; C07K 1/24

(52) U.S. Cl. CPC C07K 14/4732; A23C 19/055; A23C 20/00; A23J 1/008; C07K 1/24

(57) ABSTRACT

The invention relates to the food industry. In particular, it relates to a method for the enrichment of a casein in a composition comprising the casein and other biomolecules. It further concerns edible compositions that comprise the casein.

### FIELD OF THE INVENTION

The invention relates to the food industry and relates to a method for the enrichment of a casein in a composition comprising the casein and other biomolecules.

### SEQUENCE LISTING

The instant application contains a Sequence Listing which has been submitted electronically in ASCII text encoded as XML format and is hereby incorporated by reference in its entirety. The .xml file was created Jul. 6, 2026 and is named “P6113612WO-US_Updated.xml” and is 50.492 bytes in size. The sequence listing contained in this xml file is part of the specification and is hereby incorporated by reference herein in its entirety.

### BACKGROUND OF THE INVENTION

In 2050 the global population will be around 10 billion people. It is generally recognized that the production of food and its ingredients needs to change significantly to keep within the agreed sustainability development goals (SDGs) for the environment and climate.

Milk, and especially cow milk, is an important source of protein and is produced all around the world (total production in 2018:843 million tons). However, dairy production has an enormous impact on the environment. Currently over two-thirds of the world's agricultural land is used for maintaining livestock, including beef and dairy cows. Dairy cows and their manure generate significant amounts of greenhouse gas (including methane, which is a much more harmful greenhouse gas than CO₂) emissions which contribute to climate change. Water demand is very high as dairy operations consume large volumes of water to grow feed, water cows, manage manure and process products. Additionally, nitrogen emissions (from e.g., manure and fertilizer) cause worldwide major issues. Consequently, the carbon footprint and land-use factor of milk and cheese are high, even higher than that of pigs, fish and chicken. Next to these environmental and climatological aspects, also animal welfare is quite often compromised. Concerns about sustainability and animal-welfare of milk production are two important motivations for an increasing percentage of consumers to replace animal-based proteins by (vegan) plant-based protein sources such as soy, almond, pea and coconut.

Bovine milk contains around 35 g/L of caseins (i.e., 80% of the milk protein fraction) divided over alpha-S1-, alpha-S2-, beta- and kappa-casein within an approximate ratio of 40, 10, 40 and 10% respectively. The four caseins are well studied in terms of amino acid composition, molecular weight, post-translational modifications (PTMs) and general physico-chemical properties. Due to the high content of prolyl residues, each casein molecule has an open and flexible conformation. Furthermore, hydrophobic and hydrophilic regions show a block distribution within the protein chain, giving each casein an amphiphilic character. Because of their nature and physico-chemical properties, caseins are unique proteins that, for many applications, cannot easily be replaced by plant-based alternatives.

Expression of recombinant mammalian caseins has previously been described in non-mammalian organisms such as in yeast cells P. pastoris and S. cerevisiae (Chung, Kun-Sub, et al. Journal of Microbiology and Biotechnology 1.1 (1991): 31-36; Choi, Byung-Kwon, and Rafael Jiménez-Flores. Journal of agricultural and food chemistry 44.1 (1996): 358-364). However, the purification and recovery of the casein from the fermentation broth in which the caseins have been brought to expression is challenging and can be expensive and time consuming. It is therefore an object of the invention to provide for a method that allows easy purification of a casein in a composition comprising the casein and other biomolecules.

### SUMMARY OF THE INVENTION

In a first aspect the invention relates to a method for the enrichment of a casein in a composition comprising the casein and other biomolecules, the method comprising:
i) providing the composition comprising casein;
ii) reducing the pH of the composition to a pH lower than 3.0 so as to reduce the amount of the other biomolecules in the soluble fraction of the composition; and,
iii) recovering the soluble fraction of the composition, thereby enriching casein in the soluble fraction of the composition.

In some embodiments, step ii) comprises addition of the composition of step i) to a volume wherein the pH is maintained at a pH lower than 3.0.

In a second aspect the invention relates to a method according to claim 2, wherein the pH of the composition is instantly reduced to lower than pH 3.0 by mixing the composition into a buffered volume at a pH lower than 3.0, and wherein at least one of:
i) the composition is gradually fed into the buffered volume; and,
ii) the buffered volume is kept at a pH lower than 3.0 by feeding an acid into the buffered volume.

In a third aspect, the invention relates to a casein obtainable by the method as described herein.

In a fourth aspect the invention relates to an edible composition comprising the casein as obtained by the method as described herein.

In a fifth aspect, the invention relates to a cheese comprising predominantly a single casein and substantially no DNA.

### DETAILED DESCRIPTION OF THE INVENTION

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

For purposes of the present invention, the following terms are defined below.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 10% of the value.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (polynucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTP programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at www.ncbi.nim.nih.gov/.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. Additional factors necessary or helpful in effecting expression may also be present, such as expression enhancer elements. The expression vector will be introduced into a suitable host cell and be able to effect expression of the coding sequence in an cell culture of the host cell. The expression vector will be suitable for replication in the host cell or organism of the invention.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region, exons, introns and a 3′-nontranslated sequence (3′-end) e.g. comprising a polyadenylation- and/or transcription termination site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence May hybridize to a complementary single-stranded nucleic acid sequence. The degree of hybridization may depend on a number of factors including the amount of identity between the sequences and the hybridization conditions such as temperature and salt concentration as discussed earlier herein.

The inventors surprisingly discovered that lowering the pH of a composition comprising a casein below the isoelectric point of the casein causes the biomolecules present in the composition to precipitate while the casein remains in the soluble fraction. This allows for easy recovery of the casein.

Accordingly in a first aspect, the invention provides for a method for the enrichment of a casein in a composition comprising the casein and other biomolecules, the method comprising:
i) providing the composition comprising casein;
ii) reducing the pH of the composition to a pH lower than 3.0 so as to reduce the amount of the other biomolecules in the soluble fraction of the composition; and,
iii) recovering the soluble fraction of the composition, thereby enriching casein in the soluble fraction of the composition.

“Enrichment of a casein in a composition” is defined herein as increasing the proportion of casein in a composition comprising other biomolecules. The casein is increased in the soluble fraction, when the relative amount of casein increases as compared to the total amount of biomolecules. A casein-rich composition is a composition wherein casein represent the major protein. In a preferred embodiment, caseins represent more than 50% of proteins in said casein-rich composition. In a more preferred embodiment, caseins represent more than 80%, or 90% or 95% of proteins in said casein-rich composition. The amount of casein or other biomolecules can be measured by any method known in the art (i.e. using a gel, protein dosing).

In certain embodiments, the method of the invention can be used for the isolation and/or purification of casein from the composition comprising casein and other biomolecules.

The term “casein” is art-known and represents a family of proteins that is present in mammal-produced milk and is capable of self-assembling with other proteins in the family to form micelles and/or precipitate out of an aqueous solution at an acidic pH. Non-limiting examples of caseins include: beta-casein, kappa-casein, alpha-S1-casein, and alpha-S2-casein.

In certain embodiments, the pH in step ii) is reduced to pH 1.0 to 3.0, preferably to pH 1.5 to 2.8, more preferably to 1.8 to 2.5, or even more preferably to a pH of 2.0 to 2.4. In some embodiments the PH of the composition is reduced to 2.0. In certain embodiments, the pH can be reduced by addition of an acid to the composition. Suitable acids are preferably acids that are food safe and/or acids that are commonly used in an industrial scale such as hydrochloric acid, sulfuric acid, phosphorous acid, citric acid and acetic acid.

In certain embodiments, the composition comprising casein in step i) has a pH higher than 3.0 or at a pH higher than the isoelectric point of the casein such as for example a pH higher than 4,6. Accordingly, in certain embodiments the composition comprising casein in step i) has a pH higher than 4,6.

In an embodiment step ii) comprises adding the composition comprising the casein to a container comprising an aqueous phase having a pH of lower than 3.0, preferably having a pH between 1.0 to 3.0, preferably having a pH between 1.5 to 2.8, more preferably having a pH between 1.8 to 2.5, or even more preferably having a pH between pH of 2.0 to 2.4. During this addition the final pH of the aqueous phase is maintained by addition of acid. This addition may be accomplished by automated titration equipment or by gradually feeding the composition comprising the casein into the aqueous phase. Advantageously, according to this embodiment, most of the casein is either at neutral or at low pH whereby unwanted precipitation of casein is avoided. Accordingly, in certain embodiments, the pH of the composition comprising the casein is instantly reduced to lower than pH 3.0, 2.9, 2.8, 2.7, 2.6, 2.5, 2.4, 2.3, 2.2, 2.1, or 2.0. Therefore in certain embodiments the method of the invention comprises instantly reducing the pH of the composition comprising the casein by mixing the composition into a buffered volume at a pH lower than 3.0, and wherein at least one of:
i) the composition is gradually fed into the buffered volume; and,
ii) the buffered volume is kept at a pH lower than 3.0 by feeding an acid into the buffered volume.

In one embodiment, the other biomolecules of the composition comprise biomolecules of a host cell in which the casein was produced. A host cell as defined herein can be any suitable host cell including e.g. eukaryotic cells such as a mammalian, insect, plant, fungal, or algal cell. Preferably, however, the host cell is a microbial cell.

In certain embodiments, the other biomolecules comprise at least one of other proteins, nucleic acids, cell wall components, carbohydrates, lipo (poly) saccharides and peptidoglycans.

In certain embodiments, the casein is produced intracellularly in a host cell.

In some microbial cells, such as for example E. coli cells, recombinant proteins are often found in inclusion bodies. These intracellular particles consist essentially in aggregates of the recombinant protein. In certain embodiments, wherein the casein is produced intracellularly in a host cell, the method of the invention relates to a method of purification and/or isolation a casein in a from a composition comprising the casein and other biomolecules, the method comprising:
i) providing the composition comprising casein;
ii) reducing the pH of the composition to a pH lower less than 3.0 to extract the casein to the soluble fraction; and,
iii) isolating and/or purifying the casein from the soluble fraction of the composition.

To recover the recombinant proteins from these intracellular particles an additional thermal lysis can be performed as the soluble proteins of the host cells will precipitate or degrade at high temperature. Accordingly, in certain embodiments, the composition is heated before step ii). More specifically, the composition is subjected to a thermolysis step before step ii). In certain embodiments, the thermolysis step is performed at about 50 °C to 100 °C, preferably at about 60 °C to 90 °C.

In certain embodiments, the treatment at low pH itself may be combined with thermal lysis. Thus, step ii) may be performed at about 50 °C to 100 °C or at about 60 °C to 90 °C. The duration of such thermal lysis may be as long as the treatment at low pH but may also be shorter, for examples from 1 to 60 min, or from 2 to 30 min, or from 3 to 20 min.

In certain embodiments, an additional buffer and/or chelating agent may be present during the thermolysis steps as described herein. Suitable buffers are known to the skilled person and include for example tris(hydroxymethyl)aminomethane phosphate. Suitable chelating agents are known to the skilled person and include for example ethylenediaminetetraacetic acid, EDTA, citrate and phosphate.

In certain embodiments, the buffer and/or the chelating agent are present in a concentration from 0 to 250 mM, or preferably in a concentration from 10 to 100 mM.

In certain embodiments, the soluble fraction of step iii) is recovered by at least one of filtration, decantation, centrifugation and chromatography methods.

Chromatography methods are widely used for the purification methods, and include notably affinity chromatography, ion exchange chromatography, hydrophobic interaction chromatography and others.

Membrane filtration, including notably ultrafiltration and nanofiltration is commonly used in protein purification as well (Saxena et al. (2009) Membrane-based techniques for the separation and purification of proteins: An overview. Advances in colloids and Interface Science Volume 145, pages 1-22), and membrane filtration techniques are widely used in the dairy industry. Interestingly, they can be used to separate the different caseins from each other (see above).

It was found that raising the pH to about the isoelectric after having subjected the composition comprising casein to acidification and thereby precipitating the casein results in cheeses with improved texture.

Accordingly, in certain embodiments, the method as described herein comprises a further step iv) wherein the further step comprises raising the pH to the isoelectric point of the casein thereby precipitating the casein.

In certain embodiments, the further step comprises raising the pH to about 4.0 to 6.0, preferably the step raises the pH to about 4.5 to 5.5 to precipitate the casein. In certain embodiments, the casein is selected from the group consisting of alpha-S1-, alpha-S2-, beta- and kappa-casein and mixtures of these caseins.

As used herein, the term “alpha-S1-casein” refers to not only the alpha-S1-casein protein, but also fragments or variants thereof. Alpha-S1-casein is found in the milk of numerous different mammalian species, including cow, yak, camel, dromedary, horse, water buffalo, goat, and sheep. Of the alpha-S1-casein two variants (A-variant and B-variant) have been described.

As used herein, the term “alpha-S2-casein” refers to not only the alpha-S2-casein protein, but also fragments or variants thereof. Alpha-S2 is known as epsilon-casein in mouse, gamma-casein in rat, and casein-A in guinea pig.

As used herein, the term “beta-casein” refers to not only the beta-casein protein, but also fragments or variants thereof. For example, A1 and A2 beta-casein are genetic variants of the beta-casein milk protein that differ by one amino acid (at amino acid 67, A2 beta-casein has a proline, whereas A1 has a histidine). Other genetic variants of beta-casein include the A3, B, C, D, E, F, H1, H2, I and G genetic variants.

As used herein, the term “kappa-casein” refers to not only the kappa-casein protein, but also fragments or variants thereof. Kappa-casein is cleaved by rennet, which releases the casein macropeptide from the C-terminal region. The remaining product with the N-terminus and two-thirds of the original peptide chain is referred to as para-kappa-casein.

Preferably, the ability to express and produce the casein is conferred to the cell by transforming the cell with a nucleotide sequence coding for a casein and culturing the host cell so as to express and produce the casein. In certain embodiments, the cell has been transformed with at least one additional nucleotide sequence encoding an additional casein. In certain embodiments the cell has been transformed with a nucleotide sequence encoding at least two casein.

In some embodiments, the nucleotide sequence encodes an alpha-S1-casein and an alpha-S2-casein. In some embodiments the nucleotide sequence encodes an alpha-S1-casein and a beta-casein. In some embodiments the nucleotide sequence encodes an alpha-S2-casein and a beta-casein. In some embodiments the nucleotide sequence encodes an alpha-S1-casein and a kappa-casein. In some embodiments the nucleotide sequence encodes an alpha-S2-casein and a kappa-casein. In some embodiments the nucleotide sequence encodes a beta-casein and a kappa-casein.

Methods of transforming non-mammalian host cells are known in the art. Culture conditions and conditions conducive to the expression of casein have been described in the art and are known to the skilled person.

Non-limiting examples of sequences for casein proteins from different mammals are provided in the sequence listing herewith (see Table 1). Additional sequences for other caseins are known in the art. In addition, the caseins for use in the invention can be defined by their amino acid sequences, e.g., by comprising an amino acid sequence with a minimal percentage sequence identity to a reference casein amino acid sequence as defined herein below.

In one embodiment, the nucleotide sequence coding for a casein comprises an amino acid sequence with at least 50, 55, 60, 65, 70, 75, 80, 85, 90, 95, 96, 97, 98, 99 or 100% identity to the amino acid sequence of casein from a mammal selected from the group consisting of Bos taurus (domestic cattle), Bos grunniens (yak), Bubalus bubalis (water buffalo), Capra hircus (goat), Ovis aries (sheep), Camelus spp. (camel, dromedary), Rangifer tarandus (reindeer), Equus caballus (horse), Sus spp. including Sus domesticus (pig) and Homo sapiens.

In one embodiment, a host cell as described herein comprises at least one expression construct comprising a nucleotide sequence encoding a beta-casein comprising or consisting of an amino acid sequence with at least 50, 55, 60, 65, 70, 75, 80, 85, 90, 95, 96, 97, 98, 99, or 100% sequence identity to at least one of SEQ ID NOs 6-14.

In one embodiment, a host cell as described herein comprises at least one expression construct comprising a nucleotide sequence encoding an alpha-S1-casein comprising or consisting of an amino acid sequence with at least 50, 55, 60, 65, 70, 75, 80, 85, 90, 95, 96, 97, 98, 99, or 100% sequence identity to at least one of SEQ ID NOs: 15-23.

In one embodiment, a host cell as described herein comprises at least one expression construct comprising a nucleotide sequence encoding an alpha-S2-casein comprising or consisting of an amino acid sequence with at least 50, 55, 60, 65, 70, 75, 80, 85, 90, 95, 96, 97, 98, 99, or 100% sequence identity to at least one of SEQ ID NOs: 24-31.

In one embodiment, a host cell as described herein comprises at least one expression construct comprising a nucleotide sequence encoding a kappa-casein comprising or consisting of an amino acid sequence with at least 50, 55, 60, 65, 70, 75, 80, 85, 90, 95, 96, 97, 98, 99, or 100% sequence identity to at least one of SEQ ID NOs: 32-40.

In one embodiment, the casein that is encoded by the nucleotide sequence comprises an amino acid sequence with at least 50, 55, 60, 65, 70, 75, 80, 85, 90, 95, 96, 97, 98, 99, or 100% sequence identity to the amino acid sequence of at least one of SEQ ID NO's: 1, 2, 3, 4 and 5.

In certain embodiments, the casein is produced extracellularly in a host cell.

In certain embodiments the non-mammalian host cell is a eukaryotic host cell, preferably a eukaryotic microbial host cell, more preferably a yeast or a filamentous fungus host cell.

Filamentous fungi are herein defined as eukaryotic microorganisms that include all filamentous forms of the subdivision Eumycotina and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Examples of suitable filamentous fungal host cells includes fungi from genera Alternaria, Apophysomyces, Aspergillus, Cladosphialophora, Fonsecaea, Fusarium, Lichtheimia, Mucor, Myceliophthora, Neurospora, Penicillium, Rhizopus, Rhizomucor, Trichoderma and Trichophyton, or preferably filamentous fungi cells of the species Alternaria alternata, Apophysomyces variabilis, Aspergillus spp., Aspergillus awamori, Aspergillus foetidus, Aspergillus fumigatus, Aspergillus flavus, Aspergillus oryzae, Aspergillus niger, Aspergillus nidulans, Aspergillus sojae, Aspergillus terreus, Cladophialophora spp., Fonsecaea pedrosoi, Fusarium spp., Fusarium oxysporum, Fusarium solani, Lichtheimia spp., Lichtheimia corymbifera, Lichtheimia ramosa, Myceliophthora spp., Myceliophthora thermophila, Neurospora crassa, Penicillium chrysogenum, Penicillium simplicissimum, Penicillium brasilianum, Rhizopus spp., Rhizopus microsporus, Rhizomucor spp., Rhizomucor pusillus, Rhizomucor miehei, Trichoderma spp., Trichoderma reesei Trichophyton spp., Trichophyton interdigitale, and Trichophyton rubru, and most preferably a species selected from Aspergillus oryzae and Aspergillus niger.

Suitable strains of these filamentous fungal species are available from depository institutions known per se to the skilled person.

“Yeasts” are herein defined as eukaryotic microorganisms and include all species of the subdivision Eumycotina (Yeasts: characteristics and identification, J. A. Barnett, R. W. Payne, D. Yarrow, 2000, 3rd ed., Cambridge University Press, Cambridge UK; and, The yeasts, a taxonomic study, C P. Kurtzman and J. W. Fell (eds) 1998, 4th ed., Elsevier Science Publ. B.V., Amsterdam, The Netherlands) that predominantly grow in unicellular form. Yeasts may either grow by budding of a unicellular thallus or may grow by fission of the organism. Preferred yeasts cells for use in the present invention belong to the genera Saccharomyces, Kluyveromyces, Candida, Komagataella, Schizosaccharomyces, Hansenula, Kloeckera, Schwanniomyces, Yarrowia, Kazachstania Debaryomyces and Naumovia, or preferably yeast host cells of the species K. phaffii, K. pastoris, K. pseudopastoris S. cerevisiae, S. exiguus, S. bayanus, K. lactis, K. marxianus Y. lipolytica and S. pombe, of which K. phaffii is most preferred.

The yeast genus Pichia has more recently been reassigned to the genus Komagataella (see e.g., Heistinger et al., Microbiology, 2020; 166 (7): 614-616), which genus was split into the species K. phaffii, K. pastoris, and K. pseudopastoris. The Pichia species P. pastoris, that has been widely used in biotech industries and as used herein, has been reassigned to the Komagataella species K. phaffii (Heistinger et al., Microbiology, 2020, supra). Hence, when reference is made herein to the yeast species K. phaffii this is to be understood as equally referring to the yeast species formerly known as Pichia pastoris, and vice versa.

In certain embodiments, the non-mammalian host cell can be a prokaryotic cell, preferably a bacterial cell, including e.g. both Gram-negative and Gram-positive microorganisms. Examples of suitable bacterial host cells include host cells from the genera Escherichia, Bacillus, Lactobacillus, Lactococcus and Streptococcus, or preferably bacterial host cells of the species Escherichia coli, Bacillus subtilis, Bacillus amyloliquefaciens, Bacillus licheniformis, Bacillus coagulans, Lactobacillus acidophilus, Lactobacillus fermentum, Lactobacillus plantarum, Lactobacillus rhamnosus, Lactobacillus casei, Lactobacillus reuteri, Lactobacillus gasseri, Lactococcus lactis, Streptococcus salivarius and Streptococcus thermophilus. More preferably the bacterial cell is E. coli.

In a further aspect, the invention provides for a casein obtainable by the method as described herein.

In a further aspect, the invention provides for an edible composition comprising the casein as obtained by the method as described herein. The edible composition for human consumption comprises at least one casein as obtained by the method of the invention. In certain embodiments, the edible composition further comprises minerals, synthetic substances, flavoring substances (such as for examples herbs and spices), plant based proteins or proteins from microbial origin such as yeast proteins. Plant based proteins and yeast proteins suitable for the use in food products are known to the skilled person in the art. In certain embodiments, the edible composition is a food product. In certain embodiments the food product is a dairy product. A wide variety of dairy substitute products can be made using the methods and compositions of the present invention. Methods for producing animal-free dairy substitute products are inter alia described in WO2016/029193, which is herein incorporated by reference. Such products include without limitation, milk, whole milk, buttermilk, skim milk, infant formula, condensed milk, dried milk, evaporated milk, butter, clarified butter, cream, cottage cheese, cream cheese, creme fraiche, skyr, yogurt and various types of cheese. The dairy substitute products can also be incorporated into various food applications as a replacement for dairy products, which include ice cream, frozen custard, frozen yogurt, cookies, chocolate and cakes. In preferred embodiments, the food product is cheese.

In certain embodiments, the edible composition predominantly comprises a single casein. Relative to the total amount of caseins, the edible composition may comprise more than 50% alpha-S1-casein or more than 75% alpha-S1-casein or even more than 90% alpha-S1-casein. Relative to the total amount of caseins, the edible composition may comprise more than 50% alpha-S2-casein or more than 75% alpha-S2-casein or even more than 90% alpha-S2-casein. Relative to the total amount of caseins, the edible composition may comprise more than 50% beta-casein or more than 75% beta-casein or even more than 90% beta-casein. Relative to the total amount of caseins, the edible composition may comprise more than 50% kappa-casein or more than 75% kappa-casein or even more than 90% kappa-casein.

In yet a further aspect, the invention relates to a cheese comprising predominantly a single casein and substantially no DNA. Predominant is herein defined as comprising, relative to the total amount of caseins, at least 55%, 60%, 65%, 70%, 75%, 80%, 85%, 90%, 95%, 96%, 97%, 98%, 99%, 100% of a single casein. In some embodiments the cheese comprises at least 55%, 60%, 65%, 70%, 75%, 80%, 85%, 90%, 95%, 96%, 97%, 98%, 99%, 100% alpha-S1-casein. In some embodiments the cheese comprises at least 55%, 60%, 65%, 70%, 75%, 80%, 85%, 90%, 95%, 96%, 97%, 98%, 99%, 100% alpha-S2-casein. In some embodiments the cheese comprises at least 80%, 85%, 90%, 95%, 96%, 97%, 98%, 99%, 100% beta casein. In some embodiments the cheese comprises at least 55%, 60%, 65%, 70%, 75%, 80%, 85%, 90%, 95%, 96%, 97%, 98%, 99%, 100% kappa-casein. In some embodiments, the cheese does not comprise any beta-casein.

Substantially no DNA is herein defined as comprising no measurable traces of DNA.

Preferably, the amount of DNA is at most 0.1 wt. %, 0.01 wt. %, 0.001 wt. %, 0.0001 wt. %, preferably at most 0.00001 wt. %. In certain embodiments, the amount of DNA is between 0.00001 wt. %. and 0.1 wt. %. In certain embodiments, the amount of DNA is between 0.00001 wt. %. and 0.01 wt. %. In certain embodiments, the amount of DNA is between 0.00001 wt. %. and 0.001 wt. %. In certain embodiments, the amount of DNA is between 0.00001 wt. %. and 0.0001 wt. %.

In some embodiments, the cheese of the invention comprises no measurable traces of LPS. Preferably, the cheese of the invention comprises at most 0.1 wt. %, 0.01 wt. %, 0.001 wt. %, 0.0001 wt. %, preferably at most 0.00001 wt. % LPS.

As used herein wt. % is defined as the percentage by weight of the cheese.

In some embodiments, the cheese of the invention is a ‘fermentatively-derived bovine-like cheese’.

The present invention has been described above with reference to a number of exemplary embodiments as shown in the drawings. Modifications and alternative implementations of some parts or elements are possible and are included in the scope of protection as defined in the appended claims.

TABLE 1
Sequences

| | | SEQ |
|---|---|---|
| | | ID |
| Sequence | Species | NO |
| beta-casein | Bos taurus | 1 |
| alpha-S1-casein-Variant A | Bos taurus | 2 |
| alpha-S1-casein-Variant B | Bos taurus | 3 |
| alpha-S2-casein | Bos taurus | 4 |
| kappa-casein | Bos taurus | 5 |
| beta-casein | Ovis aries | 6 |
| beta-casein | Capra hircus | 7 |
| beta-casein | Homo sapiens | 8 |
| beta-casein | Sus domesticus | 9 |
| beta-casein | Equus caballus | 10 |
| beta-casein | Bubalus bubalis | 11 |
| beta-casein | Camelus spp | 12 |
| beta-casein | Camelus spp | 13 |
| beta-casein | Bos grunniens | 14 |
| alpha-S1-casein | Ovis aries | 15 |
| alpha-S1-casein | Capra hircus | 16 |
| alpha-S1-casein | Homo sapiens | 17 |
| alpha-S1-casein | Sus domesticus | 18 |
| alpha-S1-casein | Equus caballus | 19 |
| alpha-S1-casein | Bubalus bubalis | 20 |
| alpha-S1-casein | Camelus spp | 21 |
| alpha-S1-casein | Camelus spp | 22 |
| alpha-S1-casein | Bos grunniens | 23 |
| alpha-S2-casein | Ovis aries | 24 |
| alpha-S2-casein | Capra hircus | 25 |
| alpha-S2-casein | Sus domesticus | 26 |
| alpha-S2-casein | Equus caballus | 27 |
| alpha-S2-casein | Bubalus bubalis | 28 |
| alpha-S2-casein | Camelus spp | 29 |
| alpha-S2-casein | Camelus spp | 30 |
| alpha-S2-casein | Bos grunniens | 31 |
| kappa-casein | Ovis aries | 32 |
| kappa-casein | Capra hircus | 33 |
| kappa-casein | Homo sapiens | 34 |
| kappa-casein | Sus domesticus | 35 |
| kappa-casein | Equus caballus | 36 |
| kappa-casein | Bubalus bubalis | 37 |
| kappa-casein | Camelus spp | 38 |
| kappa-casein | Camelus spp | 39 |
| kappa-casein | Bos grunniens | 40 |

### DESCRIPTION OF THE FIGURES

FIG. 1: SDS_PAGE analysis of alpha-S1-casein produced in E. coli by fermentation at 10 L scale. SDS-PAGE analysis of 2 separated fermentation batches F10-137 and F10-138 for the expression of B variant alpha-S1-casein at 10 L scale. 1st lane Precision Plus Protein™ All Blue Prestained Protein Standards (Biorad), fermentation sample #2 after 5 h post induction and #3 after 20 h post induction, E cell-free supernatant and I intracellular cell content. F10-116 #6 intracellular content of reference batch at the end of fermentation 24 h post induction. Std Purified bovine alpha-S1-casein loaded at different amount.

FIG. 2: SDS-PAGE analysis of the extraction procedure at pH2 and 80 °C to recover the intracellular alpha-S1-casein at the end of fermentation. 1st lane Precision Plus Protein™ All Blue Prestained Protein Standards (Biorad), Purified E coli alpha-S1-casein from thermolysis at 60 °C pH8 TRIS/EDTA loaded at different amount. Fermentation sample #2 after 4 h post induction and EOF after 22 h post induction. P intracellular cell content and SN cell-free supernatant of the different step of thermolysis: Cell resuspended in water, cell suspension at pH2 before heating, cell suspension at pH 2 heated at 80 °C and cooled down to 25 °C.

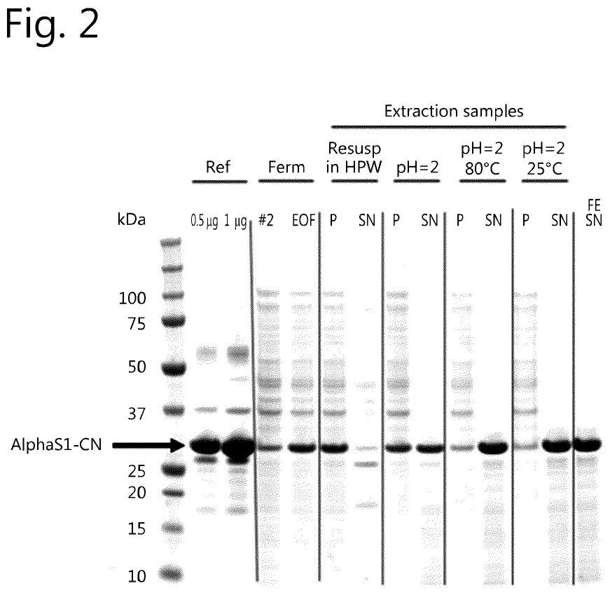

FIG. 3: A) SDS-PAGE analysis of thermolysis at pH2 using H₃PO₄ versus HCl of the production of B variant alpha-S1-casein in E. coli at 15 L scale. 1st lane Precision Plus Protein™ All Blue Prestained Protein Standards (Biorad), different samples of the extraction and further purification steps. B) picture of the sample after neutralization of the crude extract at pH8.

FIG. 4: SDS-PAGE analysis of the extraction procedure at pH 2 and pH 7 at RT to recover the intracellular alpha-S1-casein at the end of fermentation. Purified E coli alpha-S1-casein from pH 2 and pH 7 at RT at different time points of the process. Lane Std: 500 ng of purified E coli alpha-S1-Casein, Lane MW: Molecular weight Precision Plus Protein™ All Blue Prestained Protein Standards (Biorad).

### EXAMPLES

### pH Measurement

Unless otherwise mentioned, pH values were determined at 19±2 °C using a Five Easy FE20 device from Mettler Toledo equipped with an InLab Routine Pro probe. When measuring pH during thermolysis at elevated temperatures, a bioreactor equipped with an Easy Ferm Plus sensor from Hamilton was used.

### Example 1: Strain Construction and Production of Casein by Fermentation

Codon optimized bovine alpha-S1-casein genes (both variant A and B) were incorporated in the plasmid pET26b+ (Novagen). The plasmids pET26b+ containing the alpha-S1-caseins were amplified and transformed into BL21 (DE3) pLysS strain (Invitrogen). Clones resistant to kanamycin were cultivated in TB medium at 37 °C. When the OD600 nm of the culture reaches OD1, IPTG was added at 1 mM to induce the expression of the casein. Cultivation without addition of IPTG was used as negative control. The intracellular expression was evaluated on SDS-PAGE Coomassie after 7 h and 20 h induction at 37 °C (data not shown). The best clone for each casein was selected and conserved at −80 °C with 25% of glycerol for further production by fermentation. Fermentation was done in a 10 L scale bioreactor (Biostat B-plus, Sartorius) in defined minimal medium supplemented with 1% yeast extract, trace elements solution and thiamin.

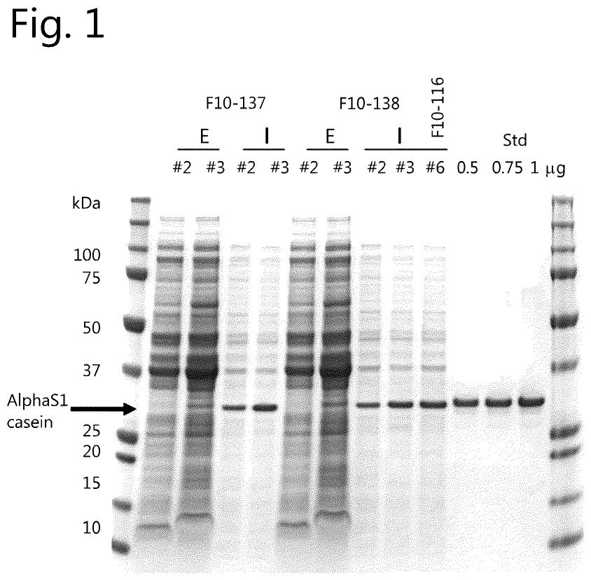

The intracellular expression of casein was confirmed by analysis of the intracellular protein content by SDS-PAGE and no expression of alpha-S1-casein was detected in the medium. Fermentation broth samples were taken before induction and after induction at different time of cultivation till end of fermentation. The cells were separated from the medium by centrifugation at 13500 rpm for 10 min at 4 °C. The cell pellets were washed with water and re-centrifuged before being diluted to obtain a suspension of 2.5 OD600 nm. 100 μl of these suspensions were treated at 100 °C for 10 min with addition of lysis buffer containing LDS and DTT. In subsequent, the total intracellular proteins extract was treated in denaturing and reducing conditions for an SDS-PAGE analysis. The cell-free supernatant (medium) was treated in the same for SDS-PAGE analysis (FIG. 1).

### Example 2: Extraction of Alpha-S1-Casein by Thermolysis at pH 2

For comparative purposes, the extraction was performed on washed cells pellet at 60 °C for 2 h in presence of TRIS buffer and EDTA at pH 8 or pH 9. The lysis of cells was very efficient to release alpha-S1-casein. However, the presence of host cell proteins contaminated the crude extract and impurities such as DNA and lipopolysaccharides (LPS) were released making the recovery of the crude extract difficult as these impurities rendered the crude extract very viscous. Moreover, the purification of such crude extract would require several steps to remove these impurities. The thermolysis of the cells pellet was further investigated at higher temperature from 70 to 90 °C at pH 8 in the presence of TRIS buffer but without EDTA to prevent major release of LPS in the crude extract. This method was also very efficient to release alpha-S1-casein and the viscosity of the crude extract was significantly reduced. Nevertheless, the quantity of DNA present in the crude extract was still high.

The thermolysis was further optimized at low pH.

Hence, at the end of the fermentation, the medium was removed by centrifugation at room temperature. The cell pellet was resuspended in water to get the same volume as before centrifugation. The pH of this suspension was 6.6 and was adjusted to pH 2.0 using concentrated phosphoric acid. The cell lysis started immediately, and alpha-S1-casein (variant B) was released in the supernatant. While maintaining a good mixing, the cell suspension at pH 2.0 was heated up to 80 °C for 2 min and then cooled down to 25 °C the cell-free crude extract was separated from the cells pellet after centrifugation and the samples were analyzed by SDS-PAGE (Error! Reference source not found.).

Overall, the above results indicate that the main quantity of intracellular alpha-S1-casein is extracted from the cells. The host cell proteins are precipitated at 80 °C and are removed after centrifugation whereas the recombinant proteins alpha-S1-casein remains in a soluble form at pH 2. The combination of the two conditions of temperature at 80 °C and acid environment at pH 2 permits the recovery and the purification alpha-S1-casein in 1 step from the E. coli cells. A total of 10 liters yielded crude B variant alpha-S1-casein having a purity above 80%.

Moreover, the gDNA, plasmid and LPS released during the extraction are strongly reduced. The lower quantity of these impurities is favorable for the next purification steps.

The determination of the total DNA content was determined by the Quant-iT™ PicoGreen™ dsDNA Assay (Invitrogen). The fermentation and extraction samples from different thermolysis procedures were analyzed to follow the reduction DNA content in the crude extract. The crude extract obtained by thermolysis at 90 °C in TRIS pH 8 (comparative experiment) was at 258 μg/mL of total DNA in average. The DNA content after thermolysis at pH 2, 80 °C was substantially reduced to 0.11 μg/mL on average.

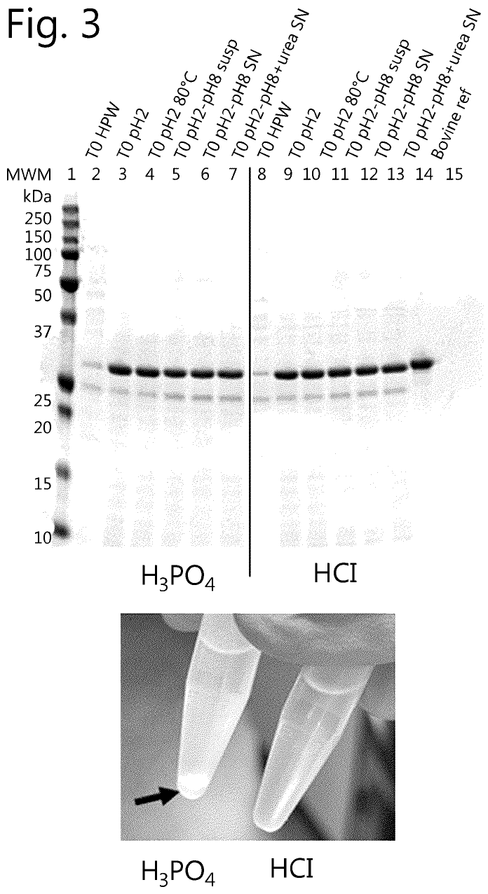

The same procedure was done using concentrated hydrochloric acid instead of phosphoric acid to adjust the pH to 2.0. A similar yield and purity of variant B alpha-S1-casein was obtained in the crude extract (FIG. 3).

### Example 3: Extraction of Alpha-S1-Casein without Thermolysis Step at pH 2 or pH7

To determine the influence of pH on the extraction efficiency of alpha-S1-casein, cells were grown as previously described herein. At the end of the fermentation, the medium was removed by centrifugation at room temperature (RT). The cell pellet was resuspended in water to get a concentration of 200 g of cell per kg. The cell pellet suspension was then divided in two parts, in one part the pH was adjusted to pH 2+/−0.2 using concentrated phosphoric acid. In the second part of the suspension the pH was kept at 7+/−0.2 using TRIS buffer.

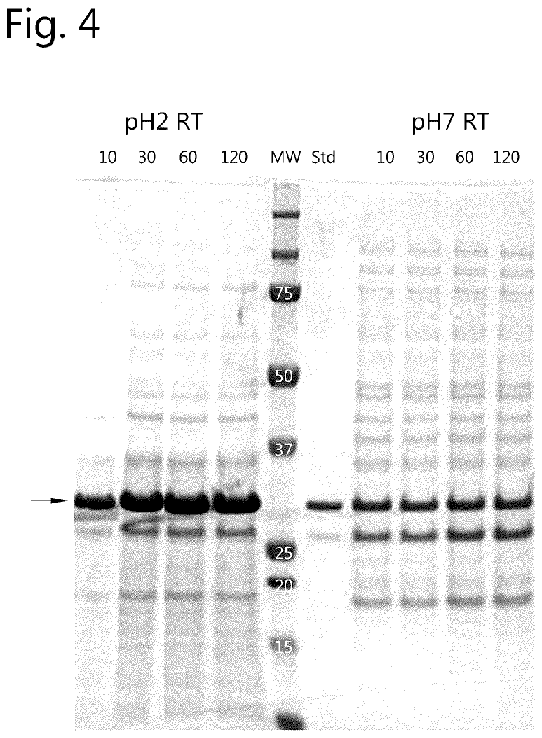

Both conditions were kept at RT while maintaining good mixing for 2 hours. Samples from both conditions were taken after 10, 30, 60 and 120 min, the cell-free crude extract was separated from the cells pellet after centrifugation and analyzed using SDS-PAGE (FIG. 4). The results showed a clear difference between extraction at pH 2 versus pH 7 at RT. Firstly, more degradation bands are observed at pH 7 than pH 2 which indicates that the lower pH protects alpha-S1-casein from proteolysis.

At pH 7 1.9 mg/mL (as quantified by UPLC-see table 2 below for full analysis) alpha-S1-casein is extracted after 10 min and this amount is not substantially increasing over time. In contrast, at pH 2, an at least 2× and up to 4× higher amount of alpha-S1-casein is obtained in the same time period.

TABLE 2
Total amount of alpha-S1-casein at RT

| Time point | | |
|---|---|---|
| sample | Total alpha-S1-casein (mg/mL) | |
| (min) | pH 2 | pH 7 |
| 10 | 6.7 | 1.9 |
| 30 | 7.2 | 2.7 |
| 60 | 7.5 | 2.9 |
| 120 | 7.6 | 3.0 |

1. A method for the enrichment of a casein in a composition comprising the casein and other biomolecules, the method comprising:
i) providing the composition comprising casein;
ii) reducing the pH of the composition to a pH lower than 3.0 so as to reduce the amount of the other biomolecules in the soluble fraction of the composition; and,
iii) recovering the soluble fraction of the composition, thereby enriching casein in the soluble fraction of the composition.

2. A method according to claim 1, wherein step ii) comprises addition of the composition of step i) to a volume wherein the pH is maintained at a pH lower than 3.0.

3. A method according to claim 2, wherein the pH of the composition is instantly reduced to lower than pH 3.0 by mixing the composition into a buffered volume at a pH lower than 3.0, and wherein at least one of:
i) the composition is gradually fed into the buffered volume; and,
ii) the buffered volume is kept at a pH lower than 3.0 by feeding an acid into the buffered volume.

4. A method according to claim 1, wherein the composition is heated before step ii).

5. A method according to claim 1, wherein the step ii) is performed at a temperature in the range of 60 °C-90 °C.

6. A method according to claim 1, wherein the method further comprises step iv) raising the pH to the isoelectric point of the casein thereby precipitating the casein.

7. The method according to claim 6, wherein the pH is raised to about 4.5 to 5.5 to precipitate the casein.

8. A method according to claim 1, wherein the casein selected from alpha-S1-, alpha-S2-, beta- and kappa-casein and mixtures of these caseins.

9. A method according to claim 1, wherein the casein is produced in a host cell, and wherein the host cell is a non-mammalian host cell that has been transformed with a nucleotide sequence coding for a casein and the non-mammalian host cell has been cultured so as to express and produce the casein.

10. The method according to claim 9, wherein the nucleotide sequence coding for a casein comprises an amino acid sequence with at least 50, 55, 60, 65, 70, 75, 80, 85, 90, 95, 96, 97, 98, 99, or 100% sequence identity to the amino acid sequence of a casein from a mammal selected from the group consisting of: Bos taurus (domestic cattle), Bos grunniens (yak), Bubalus bubalis (water buffalo), Capra hircus (goat), Ovis aries (sheep), Camelus spp. (kameel, dromedaris), Rangifer tarandus (reindeer), Equus caballus (horse), Sus spp. including Sus domesticus (pig) and Homo sapiens.

11. The method according to claim 9, wherein the nucleotide sequence encodes a casein comprising an amino acid sequence with at least 50, 55, 60, 65, 70, 75, 80, 85, 90, 95, 96, 97, 98, 99, or 100% sequence identity to the amino acid sequence of at least one of SEQ ID NO's: 1, 2, 3, 4 or 5.

12. The method according to claim 9, wherein the non-mammalian host cell is an eukaryotic host cell, preferably a eukaryotic microbial host cell, more preferably a yeast or a filamentous fungus host cell.

13. The method according to claim 12, wherein the host cell selected from a genus from the group consisting of Saccharomyces, Kluyveromyces, Candida, Komagataella, Schizosaccharomyces, Hansenula, Kloeckera, Schwanniomyces, Yarrowia, Kazachstania, Debaryomyces, Naumovia, Alternaria, Apophysomyces, Aspergillus, Cladosphialophora, Fonsecaea, Fusarium, Lichtheimia, Mucor, Myceliophthora, Neurospora, Penicillium, Rhizopus, Rhizomucor, Trichoderma and Trichophyton, wherein preferably, the cell selected from a species from the group consisting of K. phaffii, K. pastoris, K. pseudopastoris S. cerevisiae, S. exiguus, S. bayanus, Kluyveromyces lactis, Kluyveromyces marxianus Y. lipolytica, S. pombe, Alternaria alternata, Apophysomyces variabilis, Aspergillus spp., Aspergillus awamori, Aspergillus foetidus, Aspergillus fumigatus, Aspergillus flavus, Aspergillus oryzae, Aspergillus niger, Aspergillus nidulans, Aspergillus sojae, Aspergillus terreus, Cladosphialophora spp., Fonsecaea pedrosoi, Fusarium spp., Fusarium oxysporum, Fusarium solani, Lichtheimia spp., Lichtheimia corymbifera, Lichtheimia ramosa, Myceliophthora spp., Myceliophthora thermophila, Neurospora crassa, Penicillium chrysogenum, Penicillium simplicissimum, Penicillium brasilianum, Rhizopus spp., Rhizopus microsporus, Rhizomucor spp., Rhizomucor pusillus, Rhizomucor miehei, Trichoderma spp., Trichoderma reesei Trichophyton spp., Trichophyton interdigitale, and Trichophyton rubru, of which Komagataella phaffii is most preferred.

14. The method according to claim 9, wherein the non-mammalian host cell is a bacterial cell, preferably E. coli.

15. A casein obtainable by the method as described in claim 1.

16. An edible composition comprising the casein according to claim 15.

17. A cheese comprising predominantly a single casein and substantially no DNA.

18. The cheese according to claim 17, wherein the cheese comprises, relative to the total amount of caseins in the cheese, at least 55% of a single casein.


SEQUENCE LISTING (40 sequences)

SEQ ID NO: 1 - protein, 224 aa, Bos taurus
  source 1..224: note = Beta-casein
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 2 - protein, 201 aa, Bos taurus
  source 1..201: note = Alpha-S1 casein - Variant A
MKLLILTCLVAVALARPKHPIKHQGLPQPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 3 - protein, 214 aa, Bos taurus
  source 1..214: note = Alpha-S1 casein - Variant B
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 4 - protein, 222 aa, Bos taurus
  source 1..222: note = alpha-S2 casein
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 5 - protein, 190 aa, Bos taurus
  source 1..190: note = Kappa-casein
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 6 - protein, 222 aa, Ovis aries
  source 1..222: note = Beta-casein
MKVLILACLVALALAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLPPTVMFPPQSVLSLSQPKVLPVPQKAVPQRDMPIQAFLLYQEPVLGPVRGPFPILV

SEQ ID NO: 7 - protein, 222 aa, Capra hircus
  source 1..222: note = Beta-casein
MKVLILACLVALAIAREQEELNVVGETVESLSSSEESITHINKKIEKFQSEEQQQTEDELQDKIHPFAQAQSLVYPFTGPIPNSLPQNILPLTQTPVVVPPFLQPEIMGVPKVKETMVPKHKEMPFPKYPVEPFTESQSLTLTDVEKLHLPLPLVQSWMHQPPQPLSPTVMFPPQSVLSLSQPKVLPVPQKAVPQRDMPIQAFLLYQEPVLGPVRGPFPILV

SEQ ID NO: 8 - protein, 226 aa, Homo sapiens
  source 1..226: note = Beta-casein
MKVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 9 - protein, 232 aa, Sus domesticus
  source 1..232: note = Beta-casein
MKLLILACFVALALARAKEELNASGETVESLSSSEESITHISKEKIEKLKREEQQQTENERQNKIHQFPQPQPLAHPYTEPIPYPILPQNILPLAQVPVVVPLLHPEVMKDSKAKETIVPKRKGMPFPKSPAEPFVEGQSLTLTDFEVLSLPLLQSLMHQIPQPVPQTPMFAPQPLLSLPQAKVLPVPQQVVPFPQRDMPFQALLLYQDPLLGPLQGFYPVPQPVAPVYNPV

SEQ ID NO: 10 - protein, 233 aa, Equus caballus
  source 1..233: note = Beta-casein
MKILILACLVALALAREKEELNVSSETVESLSSNEPDSSSEEKLQKFKHEGQQQREVERQDKISRFVQPQPVVYPYAEPVPYAVVPQSILPLAQPPILPFLQPEIMEVSQAKETILPKRKVMPFLKSPIVPFSERQILNPTNGENLRLPVHLIQPFMHQVPQSLLQTLMLPSQPVLSPPQSKVAPFPQPVVPYPQRDTPVQAFLLYQDPRLGPTGELDPATQPIVAVHNPVIV

SEQ ID NO: 11 - protein, 224 aa, Bubalus bubalis
  source 1..224: note = Beta-casein
MKVLILACLVALALARELEELNVPGEIVESLSSSEESITHINKKIEKFQSEEQQQTEDELQDKIHPFAQTQSLVYPFPGPIPNSLPQNIPPLTQTPVVVPPFLQPEIMGVSKVKEAMAPKHKEMPFPKYPVEPFTESQSLTLTDVENLHLPLPLLQSWMHQPPQPLPPTVMFPPQSVLSLSQSKVLPVPQKAVPYPQRDMPIQAFLLYQEPVLGPVRGPFPIIV

SEQ ID NO: 12 - protein, 232 aa, Camelus sp.
  source 1..232: note = Beta-casein
MKVLILACRVALALAREKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAMPVQAVLPFQEPVPDPVRGLHPVPQPLVPVIA

SEQ ID NO: 13 - protein, 232 aa, Camelus sp.
  source 1..232: note = Beta-casein
MKVLILACLVALALAREKEEFKTAGEALESISSSEESITHINKQKIEKFKIEEQQQTEDEQQDKIYTFPQPQSLVYSHTEPIPYPILPQNFLPPLQPAVMVPFLQPKVMDVPKTKETIIPKRKEMPLLQSPVVPFTESQSLTLTDLENLHLPLPLLQSLMYQIPQPVPQTPMIPPQSLLSLSQFKVLPVPQQMVPYPQRAIPVQAVLPFQEPVPDPVRGLHPVPQPLVPVIA

SEQ ID NO: 14 - protein, 259 aa, Bos grunniens
  source 1..259: note = Beta-casein
MPLNTIYKQPQNQIIIHSAPPSLLVLYFGKKELGAMKVLILACLVALALARELEELNVPGEIVEGLSSSEESITRINKKIEKFQSEEQQQTEDGLQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 15 - protein, 206 aa, Ovis aries
  source 1..206: note = Alpha-S1 casein
MKLLILTCLVAVALARPKHPIKHQGLSPEVLNENLLRFVVAPFPEVFRKENINELSKDIGSESIEQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 16 - protein, 213 aa, Capra hircus
  source 1..213: note = Alpha-S1 casein
MKLLILTCLVAVALARPKHPINHQGLSPEVPNENLLRFVVAPFPEVFRKENINELSKDIGSESTEDQAMEDAKQMKAGSSSSSEEIVPNSAEKYIQKEDVPSERYLGYLEQLLRLKKYNVPQLETVPNSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 17 - protein, 185 aa, Homo sapiens
  source 1..185: note = Alpha-S1 casein
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 18 - protein, 206 aa, Sus domesticus
  source 1..206: note = Alpha-S1 casein
MKLLIFICLAAVALARPKPPLRHQEHLQNEPDSREELFKERKFLRFPEVPLLSQFRQEIINELNRNHGMEGHEQRGSSSSSSEEVVGNSAEQKHVQKEEDVPSQSYLGHLQGLNKYKLRQLEAIHDQELHRTNEDKHTQQGEPMKGVNQEQAYFYFEPLHQFYQLDAYPYATWYYPPQYIAHPLFTNIPQPTAPEKGGKTEIMPQW

SEQ ID NO: 19 - protein, 212 aa, Equus caballus
  source 1..212: note = Alpha-S1 casein
MKLLILTCLVAVALARPKLPHRQPEIIQNEQDSREKVLKERKFPSFALEYINELNRQRELLKEKQKDEHKEYLIEDPEQQESSSTSSSEEVVPINTEQKRIPREDMLYQHTLEQLRRLSKYNQLQLQAIHAQEQLIRMKENSQRKPMRVVNQEQAYFYLEPFQPSYQLDVYPYAAWFHPAQIMQHVAYSPFHDTAKLIASENSEKTDIIPEW

SEQ ID NO: 20 - protein, 214 aa, Bubalus bubalis
  source 1..214: note = Alpha-S1 casein
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSEKTTMPLW

SEQ ID NO: 21 - protein, 222 aa, Camelus sp.
  source 1..222: note = Alpha-S1 casein
MKLLILTCLVAVALARPKYPLRYPEVFQNEPDSIEEVLNKRKILDLAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQPFPQFFQLGASPYVAWYYPPQVMQYIAHPSSYDTPEGIASEDGGKTDVMPQWW

SEQ ID NO: 22 - protein, 230 aa, Camelus sp.
  source 1..230: note = Alpha-S1 casein
MKLLILTCLVAVALARPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 23 - protein, 210 aa, Bos grunniens
  source 1..210: note = Alpha-S1 casein
MRLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSEHYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTT

SEQ ID NO: 24 - protein, 223 aa, Ovis aries
  source 1..223: note = alpha-S2 casein
MKFFIFTCLLAVALAKHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 25 - protein, 223 aa, Capra hircus
  source 1..223: note = alpha-S2 casein
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 26 - protein, 235 aa, Sus domesticus
  source 1..235: note = alpha-S2 casein
MKFFIFTCLLAVAFAKHEMEHVSSSEESINISQEKYKQEKNVINHPSKEDICATSCEEAVRNIKEVGYASSSSSEESVDIPAENVKVTVEDKHYLKQLEKISQFYQKFPQYLQALYQAQIVMNPWDQTKTSAYPFIPTVIQSGEELSTSEEPVSSSQEENTKTVDMESMEEFTKKTELTEEEKNRIKFLNKIKQYYQKFTWPQYIKTVHQKQKAMKPWNHIKTNSYQIIPNLRYF

SEQ ID NO: 27 - protein, 231 aa, Equus caballus
  source 1..231: note = alpha-S2 casein
MKFFIFTCLLAVALAKHNMEHRSSSEDSVNISQEKFKQEKYVVIPTSKESICSTSCEEATRNINEMESAKFPTEVYSSSSSSEESAKFPTEREEKEVEEKHHLKQLNKINQFYEKLNFLQYLQALRQPRIVLTPWDQTKTGDSPFIPIVNTEQLFTSEEIPKKTVDMESTEVVTEKTELTEEEKNYLKLLYYEKFTLPQYFKIVRQHQTTMDPRSHRKTNSYQIIPVLRYF

SEQ ID NO: 28 - protein, 222 aa, Bubalus bubalis
  source 1..222: note = alpha-S2 casein
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 29 - protein, 193 aa, Camelus sp.
  source 1..193: note = alpha-S2 casein
MKFFIFTCLLAVVLAKHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 30 - protein, 193 aa, Camelus sp.
  source 1..193: note = alpha-S2 casein
MKFFIFTCLLAVVLAKHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFNKKTELTEEEKDHQKFLNKIYQYYQTFLWPEYLKTVYQYQKTMTPWNHIKRYF

SEQ ID NO: 31 - protein, 222 aa, Bos grunniens
  source 1..222: note = alpha-S2 casein
MKFFIFTCLLAVALAKNTMEHVSSSEESIISQETYKQEKNMAINPSKENLCSTFCKEVVRNANEEEYSIGSSSEESAVVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMEPWIQPKTKVIPYVRYL

SEQ ID NO: 32 - protein, 192 aa, Ovis aries
  source 1..192: note = Kappa-casein
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 33 - protein, 192 aa, Capra hircus
  source 1..192: note = Kappa-casein
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 34 - protein, 182 aa, Homo sapiens
  source 1..182: note = Kappa-casein
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPTPATEPTVDSVVTPEAFSESIITSTPETTTVAVTPPTA

SEQ ID NO: 35 - protein, 188 aa, Sus domesticus
  source 1..188: note = Kappa-casein
MMKSSFLIVPILALTLPFLGAEEQNQEKLTRCESDKRLFNEEKVKYIPIYYMLNRFPSYGFFYQHRSAVSPNRQFIPYPYYARPVVAGPHAQKPQWQDQPNVYPPTVARRPRPHASFIAIPPKKNQDKTAIPAINSIATVEPTIVPATEPIVNAEPIVNAVVTPEASSEFLITSAPETTTVQVTSPVV

SEQ ID NO: 36 - protein, 185 aa, Equus caballus
  source 1..185: note = Kappa-casein
MKSFFLVVNILALTLPFLGAEVQNQEQPTCHKNDERFFDLKTVKYIPIYYVLNSSPRYEPIYYQHRLALLINNQHMPYQYYARPAAVRPHVQIPQWQVLPNIYPSTVVRHPCPHPSFIAIPPKKLQEITVIPKINTIATVEPTPIPTPEPTVNNAVIPDASSEFIIASTPETTTVPVTSPVVQKL

SEQ ID NO: 37 - protein, 190 aa, Bubalus bubalis
  source 1..190: note = Kappa-casein
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 38 - protein, 182 aa, Camelus sp.
  source 1..182: note = Kappa-casein
MKSFFLVVTILALTLPFLGAEVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 39 - protein, 182 aa, Camelus sp.
  source 1..182: note = Kappa-casein
MKSFFLVVTILALTLPFLGAEVQNQEQPTCCEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 40 - protein, 190 aa, Bos grunniens
  source 1..190: note = Kappa-casein
MMKSFFLVVTILALTLPFLGAQEQNQEQPIRCEKDERFFSDKIAKYIPIQYVLSRYLSYGLNYYQQKPVALINNQFLPYPYYAKPAAVRSPAQILQWQVLSNTVPAKSCQAQPTTMARHPHPHLSFMAIPPKKNQDKTEIPTINTIASGERTSTPTTEAVESTVATLEASPEVIESPPEINTVQVTSTAV